(19) United States
(12) Patent Application Publication
(10) Pub. No.: US 2005/0226939 A1
(43) Pub. Date: Oct. 13, 2005

(54) Production of nano-sized hydroxyapatite particles

(75) Inventors: Murugan Ramalingam, Singapore (SG); Seeram Ramakrishna, Singapore (SG)

Correspondence Address: WILMER CUTLER PICKERING HALE AND DORR LLP, 60 STATE STREET, BOSTON, MA, 02109, US

(73) Assignee: National University of Singapore, Singapore (SG)

(21) Appl. No.: 10/819,858

(22) Filed: Apr. 7, 2004

(51) Int. Cl. A61B 5/055; A61K 33/42

(52) U.S. Cl. 424/602; 423/308

(57) ABSTRACT

Nano-sized hydroxyapatite particles are formed in a method comprising the steps of preparing a reaction solution containing a mixture of calcium ions and phosphate ions, stirring the reaction solution at a defined stirring speed, at a defined pH range and at a defined temperature range to form a suspension of hydroxyapatite seed particles, and subjecting the suspension of hydroxyapatite particles to microwave radiation for a defined time period, while maintaining the stirring speed, to form a suspension of nano-sized hydroxyapatite particles. The suspension of hydroxyapatite particles may preferably be aged for a period of time prior to the microwave radiation.

### FIELD OF INVENTION

The present invention relates to a process for forming nano-sized hydroxyapatite particles.

### BACKGROUND

Hydroxyapatite Ca₁₀(PO₄)₆(OH)₂ is structurally and chemically similar to mammalian bone. Synthetic hydroxyapatite can be used for bone implants in humans. A porous synthetic implant is implanted into and is accepted by the body. As the implant is porous, normal tissue integrates into the hydroxyapatite structure of the implant.

Hydroxyapatite can be synthesized via numerous production routes, using a range of different reactants. The most commonly used technique is the so-called ‘wet chemical’ technique, which involves precipitation of hydroxyapatite from an aqueous solution containing calcium (Ca²⁺) and phosphate (PO₄²⁻) precursors. One known wet chemical technique involves adding a solution of orthophosphoric acid at a pH greater than 9 in a dropwise manner to a dilute solution/suspension of calcium hydroxide. The orthophosphoric acid is added at a controlled rate, with stirring being maintained throughout the process. The precipitation reaction is slow and the reaction is carried out at a temperature of about 90° C. The hydroxyapatite precipitate is filtered and subsequently washed.

The quality of the hydroxyapatite synthesized is determined by its homogeneity and porosity, i.e., homogeneity in phase and low percentage of voids formed. One problem with the wet chemical technique is that the hydroxyapatite formed may contain voids which is deleterious to its mechanical strength. Accordingly, to remove the voids, an additional densification step such as sintering is often required.

Another problem with the technique is the generation of impurity phases due to the presence of unreacted calcium and phosphate precursors in the precipitation reaction. This results in the morphology of the hydroxyapatite having non-homogeneous phases or lacking in crystallinity.

Furthermore, in precipitation reactions, the particles tend to agglomerate, making it difficult to control the size of the particles.

It is desirable that hydroxyapatite for use in implants be bioresorbable so that it can be replaced, over a period of time, with regenerated bone upon implantation into the body. Currently available hydroxyapatite is typically highly stable, which significantly impedes the rate of bone regeneration when used as a hard tissue replacement material, and in alveolar ridge augmentation in particular. Furthermore, high temperature processing techniques for the production of hydroxyapatite may hinder its bioactivity.

It is thought that the solubility or bioresobability of hydroxyapatite can be enhanced by controlling one or more factors such as particle size or the phase and/or chemical homogeneity of the hydroxyapatite particles precipitated by the wet chemical method.

A need exists to provide a method of producing nano-sized hydroxyapatite particles that are bioresorbable or provide an improvement over known hydroxyapatite synthesis methods or which ameliorate at least one or more of the disadvantages referred to above.

### SUMMARY OF INVENTION

According to a first aspect of the invention, there is provided a method for producing nano-sized hydroxyapatite particles comprising:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

According to a second aspect of the invention, there is provided nano-sized hydroxyapatite particles prepared by a process comprising the steps of:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

According to a third aspect of the invention, there is provided a particulate biomaterial comprising a coherent mass of nano-sized hydroxyapatite particles prepared by a process comprising the steps of:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

According to a fourth aspect of the invention, there is provided a biomedical implant device comprising a substrate for biomedical implant into a mammal and a hydroxyapatite coating layer provided on the surface of the substrate, the hydroxyapatite coating layer formed by a method for producing hydroxyapatite nano-sized particles comprising the steps of:
(a) providing a reaction solution containing Ca²⁺ ions and PO₄³⁻ ions;
(b) stirring the reaction solution at a pH range and at a temperature to form a suspension of hydroxyapatite seed particles; and
(c) subjecting the reaction solution to microwave radiation for a period so as to form nano-sized hydroxyapatite particles.

### Definitions

The following words and terms used herein shall have the meaning indicated:

The word “biomaterial” and grammatical variations thereof is to be interpreted broadly to include any material that is biologically compatible by not producing a toxic, injurious, or immunological response in living tissue.

### BRIEF DESCRIPTION OF THE DRAWINGS

These and other features, aspects and advantages of the present invention will become better understood from the following description of non-limiting embodiments with reference to the accompanying drawings, where:

FIG. 1 shows a flow diagram of a method for producing nano-sized hydroxyapatite particles in accordance with a first embodiment of the present invention;

FIG. 2 shows a schematic diagram of a laboratory scale reactor vessel used to produced nano-sized hydroxyapatite particles in accordance with the first embodiment of the present invention.

FIG. 3 shows a flow diagram of a method for producing nano-sized hydroxyapatite particles in accordance with a second embodiment of the present invention;

FIG. 4 shows a SEM photograph of 12,000× magnification of nano-sized hydroxyapatite particles produced in accordance with the embodiments of the present invention;

FIG. 5 shows a FTIR spectrum of nano-sized hydroxyapatite particles produced in accordance with the embodiments of the present invention;

FIG. 6 shows the solubility of nano-sized hydroxyapatite particles produced in accordance with the embodiments of the present invention in simulated body fluid (SBF) medium; and

FIG. 7 shows a SEM photograph of 12,000× magnification of hydroxyapatite precipitate that has not undergone microwave radiation.

FIG. 8 shows an X-Ray Diffraction (XRD) pattern of nano-sized hydroxyapatite particles that has undergone microwave radiation.

### DETAILED DESCRIPTION OF EMBODIMENTS

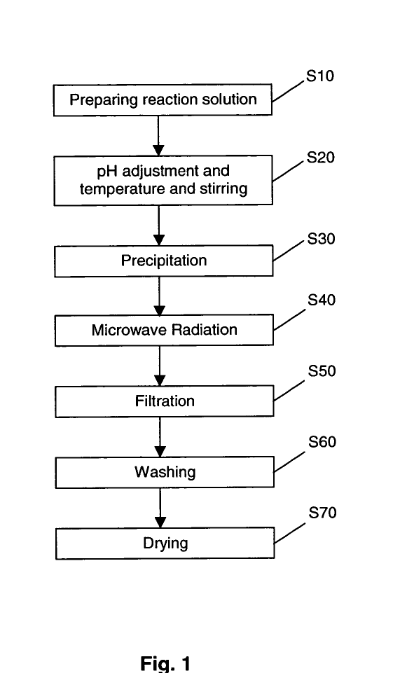

FIG. 1 shows a flow diagram of a method for producing nano-sized hydroxyapatite particles in accordance with a first embodiment of the present invention.

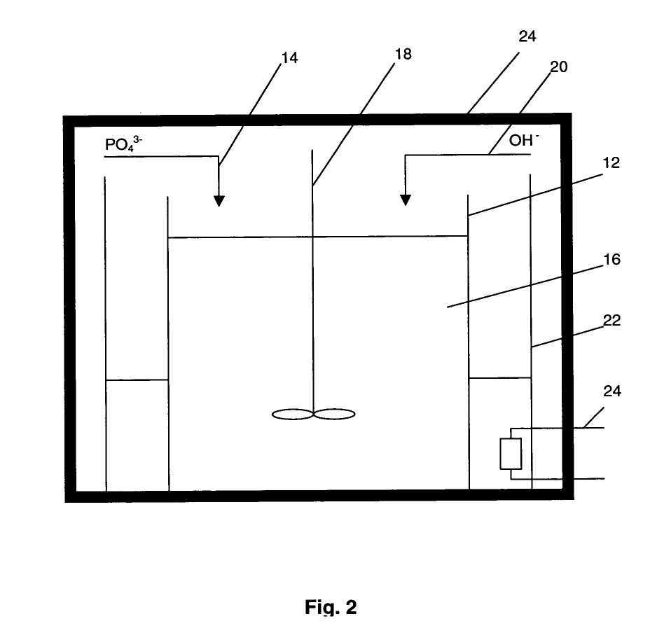

FIG. 2 shows a schematic diagram of a laboratory scale reactor vessel 12 used to produced nano-sized hydroxyapatite particles. The reactor vessel 12 is made of a non-reflecting material such as glass and is shown located within a microwave 24.

Referring to FIG. 1 and FIG. 2, the first embodiment relates to a method for forming nano-sized hydroxyapatite particles. The method involves the step of preparing a reaction solution (S10) containing a mixture of calcium ions (Ca²⁺) and phosphate ions (PO₄³⁻). As shown in FIG. 2, the reaction solution may be prepared by adding, in a dropwise manner, a solution containing a high concentration of the PO₄³⁻ ions (14) to a solution containing a low concentration of the Ca²⁺ ions (16).

Suitably, the Ca²⁺ ions (16) may be sourced from an aqueous solution selected from, but not limited to, the group consisting of: CaCl₂, CaF₂, CaBr₂, CaI₂, Ca(NO₃)₂; Ca(OH)₂; CaH₂; CaO; CaS; CaSe; CaCO₃; and one or more mixtures thereof.

Suitably the PO₄³⁻ ions (14) may be sourced from an aqueous solution containing dissolved phosphate PO₄³⁻ ions selected from, but not limited to, the group consisting of: (NH₄)₃PO₄; (NH₄)₂HPO₄, NH₄H₂PO₄, H₃PO₄, Na₃PO₄, Na₂HPO₄, NaH₂PO₄, Li₃PO₄, Li₃PO₄, Li₂HPO₄, LiH₂PO₄, K₃PO₄, K₂HPO₄, KH₂PO₄, and one or more mixtures thereof.

Suitably, as the solution containing the phosphate ions (14) may be introduced dropwise to the solution containing the calcium ions (16), the reaction solution may be stirred at a defined stirring speed, at a defined pH range and at a defined temperature range to form a suspension of hydroxyapatite seed particles (S30).

Suitably, the phosphate ions (14) may be added to the calcium ions (16) in a stoichiometric amount in order to precipitate the hydroxyapatite seed particles. Suitably the phosphate ions (14) may be added to the calcium ions (16) so that the molar ratio of Ca/P in the vessel is maintained at 1.67 or more. Suitably, the ratio of Ca/P can be maintained within the range selected from, but not limited to, the group consisting of: 1.5 to 2; 1.65 to about 1.7; 1.67 to 1.9, 1.67 to 1.80, and 1.67 to 1.76.

Advantageously, if the Ca/P molar ratio is less than 1.67, tricalcium phosphate (TCP) may form. If the Ca/P molar ratio is more than 1.67, tetra calcium phosphate (TTCP) may form. Natural bone mineral also contains substantial amount of TCP and therefore, in some embodiments, varying the Ca/P molar ratio from 1.67 may approximate natural bone constituents in the synthesized hydroxyapatite particles.

Suitably, the stirring can be achieved by locating impeller (18) in the reactant solution. This ensures that there is an even distribution of the PO₄³⁻ ions (14) within the solution of Ca²⁺ ions (16), so that a disproportionate concentration of PO₄³⁻ ions (14) in any part of the solution of Ca²⁺ ions (16) is avoided.

Suitably, the defined stirring speed may be in the range selected from, but not limited to, the group consisting of: 100 to 2500 rpm; 200 to 2300 rpm; 300 to 2100 rpm; 400 to 1900 rpm; 500 to 1700 rpm; 600 to 1500 rpm; 700 to 1300 rpm; 800 to 1200 rpm; and 900 to 1100 rpm.

Suitably, the defined pH range may be in the range selected from, but not limited to, the group consisting of: 8 to 14; 9 to 13; 9.5 to 12.5; 10 to 12; and 10 to 11. Suitably, the pH of the reaction solution may be adjusted by injecting concentrated hydroxide solution (20) into the reaction solution as it is being stirred (S20). Suitably, the concentrated hydroxide solution (20), could, for example, be NH₄OH, NaOH, KOH and one or mixtures thereof.

The vessel (12) may be located in a water bath (22) to control the temperature of reactant solution in the vessel (12). The water bath (22) is provided with a heater element (24) that is thermostatically controlled to maintain the temperature of the reactant solution.

Suitably, the defined temperature range may be in the range selected from, but not limited to, the group consisting of: 0° C. to 100° C.; 10° C. to 95° C.; 20° C. to 90° C.; 30° C. to 85° C.; 40° C. to 70° C.; and 50° C. to 65° C. In one embodiment, the defined temperature may be about 60° C.

The hydroxyapatite seed particles formed upon reaction of the calcium ions (16) and the phosphate ions (14) are suspended in the reactant solution. The hydroxyapatite seed particles may have a typical size in the range of 1 to 10 microns (μm). The suspension of hydroxyapatite seed particles is subjected to microwave radiation (S40), while maintaining the same stirring speed to form a suspension of nano-sized hydroxyapatite particles.

In one embodiment, the microwave radiation may be emitted upon introduction of the phosphate ions to the calcium ions, that is before the precipitation of the hydroxyapatite seed particles. In another embodiment, the hydroxyapatite seed particles may be first formed and then subjected to microwave radiation.

In one embodiment, the microwave radiation may be emitted continuously for a single defined time period or it may be emitted intermittently during the defined time period.

Suitably, the microwave radiation may be applied to the hydroxyapatite particles to form nano-sized grains for a time period selected from, but not limited to, the group consisting of: 1 minute to 60 minutes; 2 minutes to 55 minutes; 3 minutes to 50 minutes; 4 minutes to 45 minutes; 5 minutes to 40 minutes; 6 minutes to 35 minutes; 7 minutes to 30 minutes; 10 minutes to 25 minutes; 12 to 18 minutes. In one embodiment, the microwave radiation may be applied to the hydroxyapatite particles to form nano-sized grains for about 15 minutes.

Suitably, the nano-sized particles may be grown by emitting the microwave radiation (S40) at a frequency in the range selected from, but not limited to, the group consisting of: 1,600 to 30,000 megahertz; 2,000 to 3,500 megahertz; 2,000 to 3,000 megahertz; 2,100 to 2,800 megahertz; 2,200 to 2,600 megahertz; 2,300 to 2,550 megahertz; 2,400 to 2,500 megahertz; and 2,440 to 2,480 megahertz. In one embodiment, the nano-sized particles are grown by emitting the microwave radiation (S40) at a frequency of about 2,450 megahertz.

The source of the microwave radiation may be from a microwave oven (24), which may be an industrial microwave oven or a domestic kitchen microwave oven depending on the scale that the hydroxyapatite nano-sized particles are being produced. In the embodiment of FIG. 2, the microwave oven (24) is a domestic kitchen microwave oven.

Suitably, the power of the microwave oven (24) may be in the range selected from, but not limited to, the group consisting of: 300 W to 100,000 W; 400 W to 2,000 W; 500 W to 1,500 W; 800 W to 1,200 W; and 1000 W to 1,100 W. In one embodiment, the power of the microwave oven is about 800 W.

Suitably, the particle size of the nano-sized hydroxyapatite particles obtained may be in the range selected from, but not limited to, the group consisting of: 100 nm to 400 nm; 120 nm to 350 nm; 140 nm to 300 nm; 150 nm to 290 nm; 160 nm to 280 nm; 170 nm to 270 nm; 180 nm to 260 nm; 170 nm to 250 nm; 180 nm to 240 nm; 190 nm to 230 nm; and 200 nm to 225 nm. In one embodiment, the mean particle size of the nano-sized hydroxyapatite particles is 220 nm.

After the suspension of nano-sized hydroxyapatite particles have been formed, the particles may be subjected to a filtering step (S50). A suitable filter may be a rotary drum filter, a filter press, a vacuum filter or a bag filter so that excess reactant liquor may be washed (S60) from the particles with water to remove any by-products. After washing (S60), the nano-sized particles may be dried in an oven (S70).

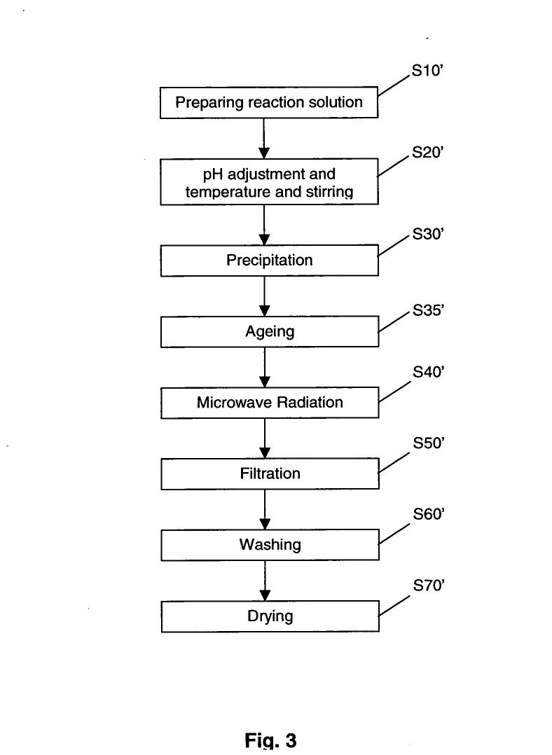

FIG. 3 shows a flow diagram of a method for producing nano-sized hydroxyapatite particles in accordance with a second embodiment of the present invention. All of the steps of the second embodiment are the same as those described above and for convenience have been marked with the same reference numeral together with the prime symbol (′). The second embodiment relates to a method for forming nano-sized hydroxyapatite particles comprising the steps of preparing a reaction solution containing a mixture of calcium ions and phosphate ions (S10′), stirring the reaction solution at a stirring speed, at a pH range and at a temperature range (S20′) to form a suspension of hydroxyapatite seed particles (S30′). With the stirring speed maintained, the suspension of hydroxyapatite particles is then aged for a period of time (S35′) and then subjected to microwave radiation (S40′) to form a suspension of nano-sized hydroxyapatite particles. The suspension of nano-sized hydroxyapatite particles is then filtered (S50′), washed to remove any by-products (S60′), and dried (S70′) to obtain dried nano-sized hydroxyapatite particles.

In accordance with the second embodiment, the method comprises the further step of aging (S35′) the suspension of hydroxyapatite particles prior to microwave radiation for a defined time period. Without being bound by theory it is thought that the step of aging assists in stimulating the reaction between the phosphate and calcium ions at molecular level to promote crystallinity or homogeneity in the morphology of the hydroxyapatite particles. In this regard, the aging step assists hydroxyapatite precipitate to undergo recrystallization and assists in removing occluded impurities, if any, and reducing crystal strain of the hydroxyapatite precipitate as the free energy of the crystal decreases. The aging step therefore promotes a more perfect crystal structure.

The defined time period for the aging step may be in the range selected from, but not limited to, the group consisting of: 1 hour to 100 hours; 1 hour to 50 hours; 1 hour to 72 hours; 2 hours to 60 hours; 3 hours to 48 hours; 4 hours to 40 hours; 5 hours to 30 hours; 6 hours to 28 hours; 8 hours to 24 hours; 18 hours to 50 hours; 24 hours to 40 hours; and 24 hours to 36 hours.

The temperature of the aging step may be in the range selected from, but not limited to, the group consisting of: 20° C. to 100° C.; 25° C. to 90° C.; 30° C. to 80° C.; 30° C. to 70° C.; 30° C. to 60° C.; 30° C. to 50° C.; 35° C. to 45° C.; and 35° C. to 40° C.

Without being bound by theory, the method according to the embodiments of the present invention utilize microwave radiation to stimulate the calcium and phosphate ions to react with each other to form or grow nano-sized hydroxyapatite particles or both. The resulting hydroxyapatite particles have high phase purity and chemical homogeneity. The nano-sized particles are also bioresorbable. Accordingly, it will be appreciated that the high phase and chemical purity of the nano-sized hydroxyapatite particles are highly suitable for use as biomaterials.

The nano-sized hydroxyapatite particles may be further processed into powder form, granular form or particulate material form by agglomerating the nano-sized hydroxyapatite particles.

The granular or powder form of hydroxyapatite may be prepared by compacting the nano-sized hydroxyapatite particles together under high pressure to form a dense form of hydroxyapatite. This is then sintered, and grounded or sieved to size, to obtain the granules or powder. The porosity of the hydroxyapatite powder granules may be controlled during an additional sintering step.

Spray drying may be used as an alternative process for the manufacture of hydroxyapatite powder. The nano-sized hydroxyapatite particles are mixed with water and about a 3% by dry weight organic binder, such as sodium carboxymethylcellulose. The slurry is then fed into a rotary spray head. The slurry forms an atomized cloud which is solidified by an opposing warm air stream to produce the hydroxyapatite powder. The hydroxyapatite powder, in the as spray dried state, is porous and friable. The hydroxyapatite powder may then be densified and stabilized by sintering and/or spray densification. The powdered form of hydroxyapatite can have an average particle size of 1 to 20 μm and may be used as a bone filler material.

The granular form of hydroxyapatite can have an average particle size of 600 to 3350 μm and can be used as a bone refiller and in drug delivery system applications.

The nano-sized hydroxyapatite particles can also be used to manufacture a particulate biomaterial component. The biomaterial component may be in the form selected from, but not limited to, the following group: blocks, discs, sheets or rings and can be used in bone research, in cell culture substrates and in thin film deposition targets.

The particulate hydroxyapatite material may have a density selected from, but not limited to, the following group of ranges: 1.5 gcm⁻³ to 2.5 gcm⁻³; 1.7 gcm⁻³ to 2.4 gcm⁻³; 1.8 gcm⁻³ to 2.3 gcm⁻³; 1.9 gcm⁻³ to 2.3 gcm⁻³; 1.9 gcm⁻³ to 2.2 gcm⁻³; and 1.9 gcm⁻³ to 2.1 gcm⁻³.

Another use for the nano-sized hydroxyapatite particles can be as a hydroxyapatite coating layer provided on the surface of a substrate in a biomedical implant device for biomedical implant into a mammal. Suitably, the substrate is a material selected from, but not limited to, the following group: titanium, titanium alloys; stainless steel alumina, zirconia, silicon nitride, silicon carbide, titanium nitride and aluminum nitride.

Suitably, the hydroxyapatite coating layer applied to the substrate is selected from, but not limited to, the following ranges: 1 μm to 3000 μm; 100 μm to 2000 μm; 200 μm to 1500 μm; 300 μm to 1200 μm; 400 μm to 1100 μm; and 500 μm to 1000 μm.

### BEST MODE & EXAMPLES

A best mode of preparing hydroxyapatite particles presently known to the applicant will now be described with reference to the following non-limiting example 1. A comparative example is also disclosed.

### Example 1

A suspension of hydroxyapatite seed particles were formed as follows:

300 ml of 0.3M aqueous ammonium hydrogen phosphate (NH₄)₂HPO₄ was added dropwise to 300 ml of 0.5M aqueous calcium chloride (CaCl₂) to form a reaction solution. The pH of the reaction solution was adjusted to 10 by adding concentrated NH₄OH solution using a syringe. The reaction solution was maintained at a constant temperature of 60° C.

A suspension of hydroxyapatite seed particles precipitated from the reaction solution. The suspension of hydroxyapatite seed particles were aged for 24 hours and then subjected to microwave radiation of frequency 2450 Hz for 15 minutes. A stirring condition of 1000 rpm and temperature of 60° C. was maintained throughout the foregoing processes.

The suspension, after undergoing microwave radiation, was filtered, washed until there is complete removal of water soluble ammonium chloride. The nano-sized hydroxyapatite particles were then dried in a vacuum oven.

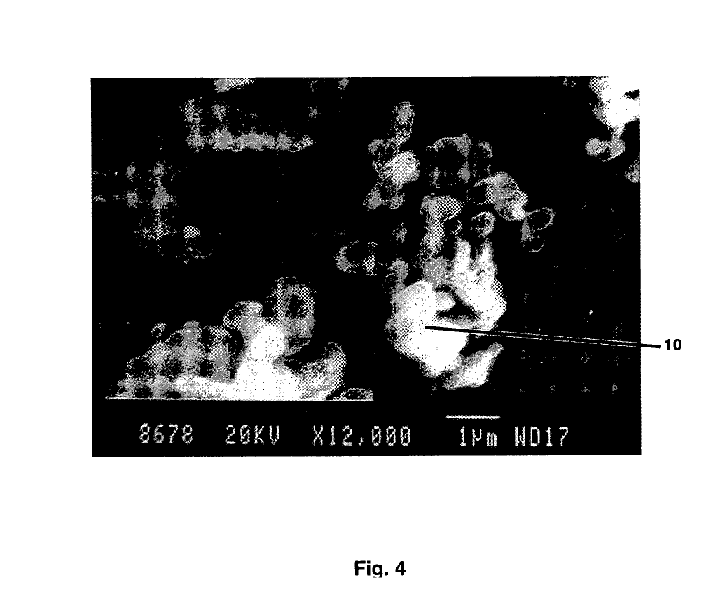

A SEM micrograph, of 12,000× magnification, of the morphology of the hydroxyapatite produced is shown in FIG. 4. The lighter areas (arrow 10) in FIG. 4 are the hydroxyapatite particles. It is clear that the size of the particles are in the nano-scale region with a mean particle size of 220 nm in diameter. Furthermore, the SEM result shows that most of the hydroxyapatite particles are not agglomerated.

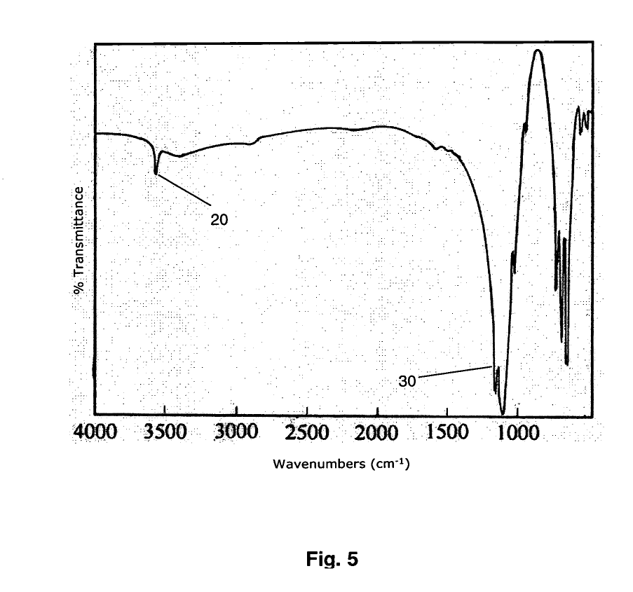

A FTIR spectroscopy spectrum showing all characteristic absorption peaks of stoichiometric hydroxyapatite obtained from the method in accordance with the embodiments of the present invention, is shown in FIG. 5. The hydroxyapatite displayed all the peaks pertaining to hydroxyl (OH⁻) and phosphate (PO₄³⁻) functional groups. The hydroxyl (OH⁻) functional groups are represented by peaks 20 and 50 whereas the phosphate (PO₄³⁻) functional groups are represented by peak 30. Hydroxyl (OH⁻) stretching vibrational band 20 and bending vibrational band 50 were observed at 3567 cm⁻¹ and 634 cm⁻¹, respectively. Major peaks of the phosphate (PO₄³⁻) group were detected at 1051 cm⁻¹, 603 cm⁻¹ and 571 cm⁻¹. A broad peak 40 relating to H₂O adsorption was noticed at 3400 cm⁻¹. No other peaks that would come from impurities or extraneous substitution of functional groups were observed. Accordingly, the spectrum established that the reaction ingredients were completely reacted and the resulting hydroxyapatite does not have any extraneous substitution. The spectrum also showed absence of impurities and hence suggests that the phase pure hydroxyapatite was produced.

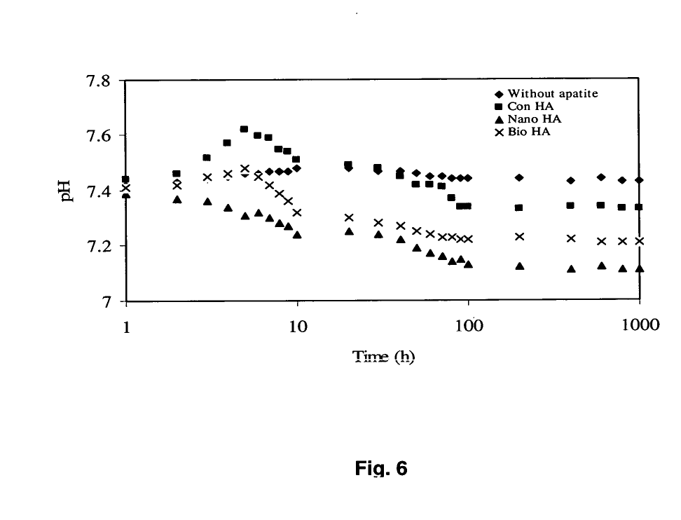

FIG. 6 shows a graph of pH vs time which illustrates the solubility of nano-sized hydroxyapatite produced in accordance with the embodiments of the present invention in simulated body fluid (SBF). The graph shows the variation in pH with time for a SBF medium (without hydroxyapatite), conventional hydroxyapatite (Con HA), nano-sized hydroxyapatite (Nano-HA) prepared from experiment 1 and biologically derived (bovine) hydroxyapatite (Bio HA). The SBF medium acts as a control sample. The pH value is dependent on solubility of the hydroxyapatite, wherein the pH decreases as the solubility increases. Accordingly, it is clear from FIG. 6 that the rate of solubility of the hydroxyapatite particles, obtained from the method in accordance with the embodiments of the present invention, was higher than conventional and biological apatites. The results suggest that the hydroxyapatite particles of the present invention have superior bioresorption which can be attributed to its high surface area to volume ratio.

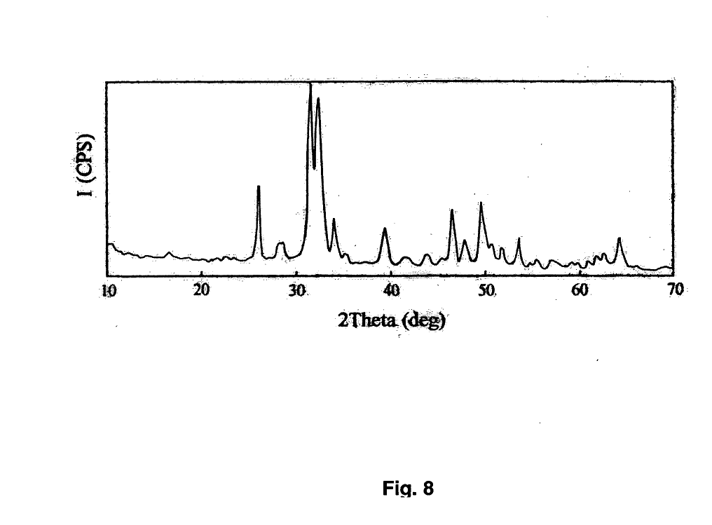

Referring to FIG. 8, there is shown an X-Ray Diffraction (XRD) pattern of nano-sized hydroxyapatite particles that has undergone microwave radiation. The XRD pattern shows broad diffracted peaks with poor crystalline nature and confirms the formation of nano-sized hydroxyapatite particle without detecting any extraneous phases. It has been found that the crystallographic behavior of nano-sized hydroxyapatite particles resembles to that of biological apatite (bioapatite). Hence, the synthesized nano-sized hydroxyapatite particles are similar in structure with naturally occurring bioapatite with respect to degree of crystallinity and structural morphology. There is also a possibility of the preparation methodology, owing to low temperature process, for getting poor crystalline nature.

The XRD pattern of FIG. 8 has been compared with XRD standard pattern data of Joint Committee on Powder Diffraction Standards (JCPDS) file number 9-432, obtained from the International Centre for Diffraction Data of Newtown Square, Pa., United States of America. The comparison showed that the synthesized hydroxyapatite particles do not have any extraneous phases other than hydroxyapatite with reference to JCPDS file 9-432. This suggests that the wet chemical reaction has produced phase pure or homogeneous hydroxyapatite. The result further supports the results of the FTIR spectrum analysis of FIG. 5.

### Comparative Example

To form a suspension of hydroxyapatite seed particles, 300 ml of aqueous ammonium hydrogen phosphate (NH₄)₂HPO₄ of concentration of 0.3M was added dropwise to 300 ml of aqueous calcium chloride (CaCl₂) of concentration 0.5 M to form a reaction solution, and the pH of the reaction solution adjusted to 10 by adding concentrated NH₄OH solution using a syringe. The suspension of hydroxyapatite was then aged for 24 hours. A stirring condition of 1000 rpm and temperature of 60° C. was maintained throughout the foregoing processes. The suspension was then filtered, washed until there is complete removal of water soluble ammonium chloride and then dried in a vacuum oven.

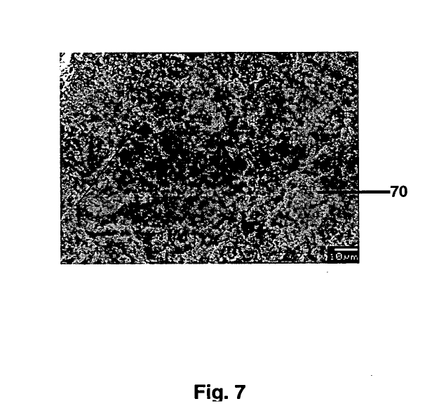

A SEM micrograph, of 12,000× magnification, of the morphology of the hydroxyapatite produced but without exposure to microwave radiation is shown in FIG. 7. The lighter areas 70 in FIG. 7 represents the hydroxyapatite particles. As can be seen from the photograph, without microwave radiation, the hydroxyapatite formed agglomerates forming voids and do not form individual nano-sized particles like in the previous example.

Effect of Aging Time

The effect of aging time on the formation of hydroxyapatite was studied by keeping the parameters of experiment 1 constant and varying the aging time.

As aging time is responsible for the recrystallization of the nano-sized hydroxyapatite particles, the crystallinity changes with respect to various aging times was studied and the results are tabulated in Table 1 below.

TABLE 1

| Aging Time (h) | HA Crystallite Size (nm) |
|---|---|
| 24 | 69 |
| 50 | 63 |
| 100 | 59 |

The results indicate that upon increasing the aging time from 24 to 100 hours, the crystallite size of the nano-sized hydroxyapatite particles decreased. This indicates that the nano-sized hydroxyapatite particles recrystallized during the aging step.

Effect of Aging Temperature

The effect of aging temperature on the formation of nano-sized hydroxyapatite particles was studied by keeping the parameters of experiment 1 constant and varying the aging temperature. As aging temperature is responsible for the crystal growth of nano-sized hydroxyapatite particles, the crystal growth changes with respect to various aging temperatures was studied and the results are tabulated in Table 2 below.

TABLE 2

| Aging Temp. (° C.) | HA Crystallite Size (nm) |
|---|---|
| 37 | 61 |
| 60 | 67 |
| 100 | 72 |

The results indicate that upon increasing the aging temperature from 37 to 100° C., the crystallite size of the nano-sized hydroxyapatite particles reduced. This indicated that the crystal growth increased with increasing temperature. Accordingly, the results suggest that crystal size of the nano-sized hydroxyapatite particles can be manipulated by controlling the reaction temperature.

### Applications

An advantage of the embodiment of the present invention is that there is no need for a sintering step, thereby saving time and costs. Conventional wet chemical methods require the resulting precipitate of hydroxyapatite particles to undergo sintering in order to densify the particles. The present invention utilizes microwave radiation combined with the precipitation reaction to obtain the nano-size hydroxyapatite particles that do not require the additional sintering step. It will be appreciated that significant time and infrastructure savings can be achieved by avoiding an additional sintering step, particularly in industrial scale plants for synthetic hydroxyapatite production.

Accordingly, as the present invention provides a simpler method for producing hydroxyapatite bioceramics, the method of the present invention is more economical compared.

Another advantage of the present invention is that the hydroxyapatite particles are bioresorbable owing to their nano-sized particles, high surface area to volume ratio, phase purity and chemical homogeneity. In this regard and without being bound by theory, it is thought that the microwave radiation assists in the growth of hydroxyapatite layers on the hydroxyapatite seed particles at the atomic level, thereby resulting in nano-sized particle having a highly homogenous phase. A nano-sized particle having high surface reactivity is obtain resulting in better interaction with living cells and tissues during bone regeneration over that of the prior art.

The highly resorbable nano-sized hydroxyapatite is useful for the formation of new mammalian bone.

Another advantage of the invention is that the method of the present invention does not result in any undesirable by-products.

It should also be realized that the nano-sized hydroxyapatite particles can be applied to applications other than as an orthopedic and dental filling biomaterial. For example, the nano-sized hydroxyapatite particles could be used in drug delivery, biomolecular delivery, proteins purification and biosensors. Accordingly, it will be appreciated that the invention is not limited to the embodiments described herein and additional embodiments or various modifications may be derived from the application of the invention by a person skilled in the art without departing from the scope of the invention.

1. A method for producing nano-sized hydroxyapatite particles comprising:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

2. The method according to claim 1 wherein the Ca²⁺ ions are sourced from an aqueous solution selected from the group consisting of CaCl₂; CaF₂; CaBr₂; CaI₂; Ca(NO₃)₂; Ca(OH)₂; CaH₂; CaO; CaS; CaSe, CaCO₃; and one or more mixtures thereof.

3. The method according to claim 1 wherein the PO₄³⁻ ions are sourced from an aqueous solution selected from the group consisting of (NH₄)₃PO₄; (NH₄)₂HPO₄; NH₄H₂PO₄; H₃PO₄; Na₃PO₄; Na₂HPO₄; NaH₂PO₄; Li₃PO₄; Li₃PO₄; Li₂HPO₄; LiH₂PO₄; K₃PO₄; K₂HPO₄; KH₂PO₄.

4. The method according to claim 1 wherein the pH is obtainable by adding hydroxide selected from the group consisting of: NH₄OH, KOH, NaOH and one or more mixtures thereof to the reaction solution.

5. The method according to claim 1 wherein a ratio of Ca/P in the reaction solution in step (b) is maintained within the range 1.5 to 2.

6. The method according to claim 5 wherein a ratio of Ca/P in the reaction solution in step (b) is maintained in the range about 1.65 to about 1.7.

7. The method according to claim 6 wherein a ratio of Ca/P in the reaction solution in step (b) is maintained at about 1.67.

8. The method according to claim 1 wherein the subjecting is carried out at a microwave radiation frequency of 1,600 to 30,000 megahertz.

9. The method according to claim 1 wherein the subjecting is carried out in an oven at a microwave radiation power of 300 W to 3000 W.

10. The method according to claim 1 wherein the providing of step (a) comprises adding a solution containing phosphate ions dropwise to a solution containing calcium ions.

11. The method according to claim 1 further comprising the step of aging the suspension of hydroxyapatite seed particles before step (c) for a period of time.

12. The method according to claim 1 wherein step (c) comprises subjecting the suspension to microwave radiation for a period from 1 to 72 hours.

13. The method according to claim 1 wherein step (b) comprises stirring the reaction solution at a stirrer speed in the range of from 100 to 2,500 rpm.

14. The method according to claim 1 wherein step (b) comprises stirring the reaction solution at a temperature in the range of from greater than 0° C. to 100° C.

15. The method according to claim 1 wherein step (b) comprises stirring the reaction solution at a pH in the range of from 8 to 14.

16. The method according to claim 1 further comprising the step of filtering the nano-sized hydroxyapatite particles from the reaction solution after step (c).

17. The method according to claim 16 further comprising the step of washing the nano-sized hydroxyapatite particles after the filtering step.

18. The method according to claim 17 further comprising the step of drying the nano-sized hydroxyapatite particles after the washing step.

19. The method according to claim 1 comprising subjecting the suspension to microwave radiation for a period so as to form nano-sized hydroxyapatite particles having diameters in the range of from 100 μm to 400 μm.

20. Nano-sized hydroxyapatite particles prepared by a process comprising the steps of:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

21. A particulate biomaterial comprising a coherent mass of nano-sized hydroxyapatite particles prepared by a process comprising the steps of:
(a) providing a reaction solution containing Ca²⁺ ions and PO₄³⁻ ions;
(b) stirring the reaction solution at a pH and at a temperature to form a suspension of hydroxyapatite seed particles; and
(c) subjecting the suspension to microwave radiation for a period so as to form nano-sized hydroxyapatite particles.

22. The particulate biomaterial according to claim 21 having a block form; or
granular form or powder form.

23. The particulate biomaterial according to claim 21 having a density in the range from 1.5 gcm⁻³ to 2.5 gcm⁻³.

24. A biomedical implant device comprising a substrate for biomedical implant into a mammal and a hydroxyapatite coating layer provided on the surface of the substrate, the hydroxyapatite coating layer formed by a method for producing hydroxyapatite nano-sized particles comprising the steps of:
(a) providing a reaction solution containing Ca²⁺ ions and PO₄³⁻ ions;
(b) stirring the reaction solution at a pH range and at a temperature to form a suspension of hydroxyapatite seed particles; and
(c) subjecting the reaction solution to microwave radiation for a period so as to form nano-sized hydroxyapatite particles.

25. The biomedical implant device according to claim 24, wherein the substrate is a material selected from the group consisting of titanium, titanium alloys; stainless steel alumina, zirconia, silicon nitride, silicon carbide, titanium nitride and aluminum nitride.

26. The biomedical implant device according to claim 24, wherein the hydroxyapatite coating layer substrate has a thickness of from 1 μm to 3000 μm.
